(19) United States
(12) Patent Application Publication
(10) Pub. No.: US 2024/0173134 A1
(43) Pub. Date: May 30, 2024

(54) Candidate Risk Assessment for a Mitral Transcatheter Edge-To-Edge Repair Procedure

(71) Applicant: Evalve, Inc., Santa Clara, CA (US)

(72) Inventors: Pavan Karlapudi, Mumbai (IN); Atif Momin, Malegaon (IN)

(73) Assignee: Evalve, Inc., Santa Clara, CA (US)

(21) Appl. No.: 18/517,102

(22) Filed: Nov. 22, 2023

(30) Foreign Application Priority Data
Nov. 24, 2022 (IN) 202211067641

(51) Int. Cl. A61F 2/24; G16H 20/40; G16H 50/20; G16H 50/30; G16H 50/70

(52) U.S. Cl. CPC A61F 2/2466; A61F 2/2427; A61F 2/2454; G16H 20/40; G16H 50/20; G16H 50/30; G16H 50/70

(57) ABSTRACT

Techniques are provided for candidate risk assessment for a mitral TEER procedure. A set of candidate anatomical features corresponding to a candidate for a mitral transcatheter edge-to-edge repair (TEER) procedure is received. A candidate feature vector based on the set of candidate anatomical features is generated. A classification engine is applied to the candidate feature vector, wherein the classification engine is trained to evaluate mitral TEER outcome. An outcome metric corresponding to a predicted mitral TEER success or a predicted mitral TEER failure for the candidate based on applying the classification engine is provided.

### CROSS-REFERENCE TO RELATED APPLICATIONS

The present application claims priority from Indian Patent Application No. 202211067641, filed Nov. 24, 2022, the disclosure of which is hereby incorporated herein by reference.

### FIELD OF THE DISCLOSURE

The present disclosure generally relates to techniques for treatment selection for a cardiac condition and relates more specifically to systems and methods for predicting procedural success for a transcatheter edge-to-edge repair (TEER) procedure.

### BACKGROUND

TEER is a minimally invasive procedure typically performed to treat mitral regurgitation (MR), which is a condition characterized by the backflow of blood through the mitral valve. During a TEER procedure, a thin, flexible catheter carrying a fixation device, such as a clip, may be percutaneously guided into the heart where it may be deployed to capture and join opposing edges of the mitral valve leaflets at the regurgitant area. The mitral valve leaflets may be repaired by creating an artificial “edge-to-edge” connection which may produce a double-orifice configuration to help improve valve coaptation or closure of the leaflets, reduce the backflow of blood, and improve overall valve function. TEER is a less invasive alternative to traditional surgical mitral valve repair or replacement. Patient selection and evaluation for TEER is an important aspect to the success of a TEER procedure. The procedure's suitability often depends on the specific characteristics of the patient's valve anatomy, severity of regurgitation, and other individual factors.

TEER has emerged as an important therapeutic option for patients with symptomatic primary or secondary mitral regurgitation (MR). Since commercial approval, improvements in procedural technique, device design, and cardiac imaging have progressively expanded the patient population that can be treated with mitral TEER. Procedural success is an important factor impacting patient outcomes following mitral TEER. However, the specific characteristics that contribute to procedural success are not well characterized and have been proven to be difficult to predict in advance of the procedure.

### BRIEF SUMMARY OF THE DISCLOSURE

In accordance with aspects of the disclosed subject matter, a computer-implemented method, which may be performed by one or more processors, may include receiving a set of candidate anatomical features corresponding to a candidate for a mitral transcatheter edge-to-edge repair (TEER) procedure, generating a candidate feature vector based on the set of candidate anatomical features, and applying a classification engine to the candidate feature vector. The classification engine may be trained to evaluate mitral TEER outcome. The computer-implemented method may also include providing an outcome metric corresponding to a predicted mitral TEER success or a predicted mitral TEER failure for the candidate based on applying the classification engine.

Additionally, in various examples, the classification engine may be a binary classification engine that may comprise a logistic regression model. The classification engine may accept an input feature that may comprise a first principal component input generated based on a regurgitant volume and an effective regurgitant orifice area. The classification engine may include a first weight corresponding to the first principal component input, and the set of candidate anatomical features may include a regurgitant volume of the candidate and an effective regurgitant orifice area of the candidate. The candidate feature vector may include a first principal component input value generated based on the regurgitant volume of the candidate and the effective regurgitant orifice area of the candidate.

Furthermore, in various examples, the classification engine may accept an input feature that may comprise a second principal component input generated based on a mitral valve peak velocity and a mean mitral gradient. The classification engine may include a second weight corresponding to the second principal component input, and the set of candidate anatomical features may include a mitral valve peak velocity of the candidate and a mean mitral gradient of the candidate. The candidate feature vector may include a second principal component input value generated based on the mitral valve peak velocity of the candidate and the mean mitral gradient of the candidate.

Also, in various examples, the classification engine may accept one or more input features that may comprise at least one of: presence of a wide jet, severe tricuspid regurgitation, and bileaflet flail prolapse. The classification engine may include one or more weights corresponding to at least one of: presence of a wide jet, severe tricuspid regurgitation, and bileaflet flail prolapse. The candidate feature vector may include at least one of: presence of a wide jet of the candidate, severe tricuspid regurgitation of the candidate, and bileaflet flail prolapse of the candidate.

Additionally, in various examples, the classification engine may be trained based on input training data that may comprise patient anatomical features before a mitral TEER procedure for a plurality of patients. The patient anatomical features may include anatomical features determined based on echocardiographic data for the plurality of patients. The patient anatomical features may include a regurgitant volume, an effective regurgitant orifice area, a mitral valve peak velocity, a mean mitral gradient, presence of a wide jet, severe tricuspid regurgitation, and bileaflet flail prolapse for the plurality of patients. The classification engine may be trained based on output training data that may comprise a determination of mitral TEER success or mitral TEER failure for the plurality of patients. Also, the classification engine may be trained based on output training data generated based on at least one of: occurrence of one or more major adverse events after the mitral TEER procedure for the plurality of patients, a mitral gradient after the mitral TEER procedure for the plurality of patients, and a mitral regurgitation grade after the mitral TEER procedure for the plurality of patients.

Further in accordance with additional aspects of the disclosed subject matter, a method for treating mitral valve regurgitation may include performing a computer implemented method in a manner as described above. If the outcome metric predicts mitral TEER success, the method for treating mitral valve regurgitation may include performing a TEER procedure on the candidate. The TEER procedure may comprise delivering an implantable fixation device to the mitral valve and grasping first and second leaflets of the mitral valve with respective first and second clamps of the implantable fixation device.

Additionally, in various examples, if the outcome metric predicts mitral TEER failure, the method of treatment may include performing an alternative procedure on the candidate. The alternative procedure may comprise securing a mitral valve prosthesis at least within an annulus of the mitral valve. Additionally, the securing step may include positioning the mitral valve prosthesis, which may have a plurality of replacement valve leaflets and a strut frame, within at least the mitral valve annulus, and deploying the mitral valve prosthesis by expanding the strut frame and the plurality of replacement valve leaflets coupled thereto. Also, the step of performing the TEER procedure may further include receiving, at the processor, post-TEER data based on an outcome of the TEER procedure, and updating, via the processor, the classification engine based on the post-TEER data. The post-TEER data of the candidate may include at least one of: occurrence of one or more major adverse events, a mitral valve gradient, a mitral regurgitation grade, and mitral TEER success or failure.

### BRIEF DESCRIPTION OF THE DRAWINGS

FIG. 1 illustrates a block diagram of an example computer system in accordance with an aspect of the disclosure.

FIG. 2A illustrates a block diagram of an example process for building, training, and evaluating a classification model.

FIG. 2B illustrates example feature selection data for a logistic regression model including principal component features.

FIG. 2C illustrates example evaluation data for a logistic regression model.

FIG. 2D illustrates example evaluation data for a logistic regression model.

FIG. 3 illustrates a flow diagram of an example process for candidate risk assessment for a mitral TEER procedure.

FIG. 4A depicts an exemplary implantable device which may be used in a TEER procedure where a TEER procedure is indicated.

FIG. 4B depicts an alternative exemplary implantable device which may be used in an alternative procedure where a TEER procedure is contraindicated.

FIG. 5 is a block diagram that illustrates a computer system upon which one or more examples may be implemented.

### DETAILED DESCRIPTION

In the following description, numerous specific details are set forth in order to provide a thorough understanding of the disclosure. It will be apparent, however, that the embodiments may be practiced without these specific details. The detailed description that follows describes exemplary embodiments and the features disclosed are not intended to be limited to the expressly disclosed combination(s). Therefore, unless otherwise noted, features disclosed herein may be combined to form additional combinations that were not otherwise shown for purposes of brevity. Additionally, while the exemplary embodiments of the present disclosure may be described in the context of a mitral valve and procedures thereof, it should be understood that such embodiments are merely exemplary and may be applicable to other heart valves (e.g., the tricuspid valve) and procedures thereof.

It will be further understood that: the term “or” may be inclusive or exclusive unless expressly stated otherwise; the term “set” may comprise zero, one, or two or more elements; the terms “first”, “second”, “certain”, and “particular” are used as naming conventions to distinguish elements from each other, and does not imply an ordering, timing, or any other characteristic of the referenced items unless otherwise specified; the term “and/or” as used herein refers to and encompasses any and all possible combinations of one or more of the associated listed items; that the terms “comprises” and/or “comprising” specify the presence of stated features, but do not preclude the presence or addition of one or more other features.

A “computer system” refers to one or more computers, such as one or more physical computers, virtual computers, and/or computing devices. For example, a computer system may be, or may include, one or more server computers, desktop computers, laptop computers, mobile devices, special-purpose computing devices with a processor, cloud-based computers, cloud-based clusters of computers, virtual machine instances, and/or other computing devices. A computer system may include another computer system, and a computing device may belong to two or more computer systems. Any reference to a “computer system” may mean one or more computers, unless expressly stated otherwise. When a computer system performs an action, the action is performed by one or more computers of the computer system.

A “device” may be a computer system, hardware, and/or software stored in, or coupled to, a memory and/or one or more processors on one or more computers. As an alternative or addition, a device may comprise specialized circuitry. For example, a device may be hardwired or persistently programmed to support a set of instructions to perform the functions discussed herein. A device may be a standalone component, work in conjunction with one or more other devices, contain one or more other devices, and/or belong to one or more other devices.

A “component” may be hardware and/or software stored in, or coupled to, a memory and/or one or more processors on one or more computers. As an alternative or addition, a component may comprise specialized circuitry. For example, a component may be hardwired and/or persistently programmed with a set of instructions to perform the functions discussed herein. A component may be a standalone component, work in conjunction with one or more other components, contain one or more other components, and/or belong to one or more other components.

### General Overview

The present disclosure generally describes techniques for candidate risk assessment for a mitral TEER procedure. A risk assessment system uses a classification engine to predict procedural success for mitral TEER at the individual patient level. The classification engine includes a classification model that is trained based on historic patient data for a plurality of patients that have undergone a mitral TEER procedure. The patient data includes anatomical features that are routinely collected during echocardiographic evaluation of patients with symptomatic primary or secondary mitral regurgitation. The risk assessment system may be used to evaluate candidates for the treatment of mitral regurgitation.

One aspect of the disclosure is directed to a computer-implemented method comprising: receiving a set of candidate anatomical features corresponding to a candidate for a mitral TEER procedure; generating a candidate feature vector based on the set of candidate anatomical features; applying a classification engine to the candidate feature vector, wherein the classification engine is trained to evaluate mitral TEER outcome; and providing an outcome metric for the candidate based on applying the classification engine; wherein the method is performed by one or more processors.

In some examples, the classification engine is a binary classification engine comprising a logistic regression model. As an alternative or addition, the outcome metric may correspond to a predicted mitral TEER success or a predicted mitral TEER failure.

In some examples, the classification engine accepts an input feature comprising a first principal component input generated based on a regurgitant volume and an effective regurgitant orifice area. As an alternative or addition, the classification engine may include a first weight corresponding to the first principal component input. As an alternative or addition, the set of candidate anatomical features may include a regurgitant volume of the candidate and an effective regurgitant orifice area of the candidate. As an alternative or addition, the candidate feature vector may include a first principal component input value generated based on the regurgitant volume of the candidate and the effective regurgitant orifice area of the candidate.

In some examples, the classification engine accepts an input feature comprising a second principal component input generated based on a mitral valve peak velocity and a mean mitral gradient. As an alternative or addition, the classification engine may include a second weight corresponding to the second principal component input. As an alternative or addition, the set of candidate anatomical features may include a mitral valve peak velocity of the candidate and a mean mitral gradient of the candidate. As an alternative or addition, the candidate feature vector may include a second principal component input value generated based on the mitral valve peak velocity of the candidate and the mean mitral gradient of the candidate.

In some examples, the classification engine accepts one or more input features comprising at least one of presence of a wide jet, severe tricuspid regurgitation, and bileaflet flail prolapse. As an alternative or addition, the classification engine may include one or more weights corresponding to at least one of presence of a wide jet, severe tricuspid regurgitation, and bileaflet flail prolapse. As an alternative or addition, the candidate feature vector may include at least one of presence of a wide jet of the candidate, severe tricuspid regurgitation of the candidate, and bileaflet flail prolapse of the candidate.

In some examples, the classification engine is trained based on input training data comprising patient anatomical features before a mitral TEER procedure for a plurality of patients. As an alternative or addition, the patient anatomical features may include anatomical features determined based on echocardiographic data for the plurality of patients. As an alternative or addition, the patient anatomical features may include a regurgitant volume, an effective regurgitant orifice area, a mitral valve peak velocity, a mean mitral gradient, presence of a wide jet, severe tricuspid regurgitation, and bileaflet flail prolapse for the plurality of patients. As an alternative or addition, the classification engine may be trained based on output training data comprising a determination of mitral TEER success or mitral TEER failure for the plurality of patients. As an alternative or addition, the classification engine may be trained based on output training data generated based on at least one of occurrence of one or more major adverse events after the mitral TEER procedure for the plurality of patients, a mitral gradient after the mitral TEER procedure for the plurality of patients, and a mitral regurgitation grade after the mitral TEER procedure for the plurality of patients.

In some examples, the classification engine is trained based on input training data comprising patient anatomical features after a mitral TEER procedure for a candidate. As an alternative or addition, the patient anatomical features may include anatomical features determined based on echocardiographic data for the candidate taken post-TEER procedure. As an alternative or addition, the patient anatomical features may include a regurgitant volume, an effective regurgitant orifice area, a mitral valve peak velocity, a mean mitral gradient, presence of a wide jet, severe tricuspid regurgitation, and bileaflet flail prolapse for the candidate post-TEER procedure. As an alternative or addition, the classification engine may be trained based on output training data comprising a determination of mitral TEER success or mitral TEER failure for candidate. As an alternative or addition, the classification engine may be trained based on output training data generated based on at least one of occurrence of one or more major adverse events after the mitral TEER procedure for the candidate, a mitral gradient after the mitral TEER procedure for candidate, and a mitral regurgitation grade after the mitral TEER procedure for the candidate.

One aspect of the disclosure is directed to a computer system including one or more hardware processors and at least one memory storing one or more instructions. The instructions, when executed by the one or more hardware processors, cause the one or more hardware processors to receive a set of candidate anatomical features corresponding to a candidate for a mitral TEER procedure; generate a candidate feature vector based on the set of candidate anatomical features; apply a classification engine to the candidate feature vector, wherein the classification engine is trained to evaluate mitral TEER outcome; and provide an outcome metric for the candidate based on applying the classification engine.

In some examples, the classification engine is a binary classification engine comprising a logistic regression model. As an alternative or addition, the outcome metric may correspond to a predicted mitral TEER success or a predicted mitral TEER failure.

In some examples, the classification engine accepts an input feature comprising a first principal component input generated based on a regurgitant volume and an effective regurgitant orifice area. As an alternative or addition, the classification engine may include a first weight corresponding to the first principal component input. As an alternative or addition, the set of candidate anatomical features may include a regurgitant volume of the candidate and an effective regurgitant orifice area of the candidate. As an alternative or addition, the candidate feature vector may include a first principal component input value generated based on the regurgitant volume of the candidate and the effective regurgitant orifice area of the candidate.

In some examples, the classification engine accepts an input feature comprising a second principal component input generated based on a mitral valve peak velocity and a mean mitral gradient. As an alternative or addition, the classification engine may include a second weight corresponding to the second principal component input. As an alternative or addition, the set of candidate anatomical features may include a mitral valve peak velocity of the candidate and a mean mitral gradient of the candidate. As an alternative or addition, the candidate feature vector may include a second principal component input value generated based on the mitral valve peak velocity of the candidate and the mean mitral gradient of the candidate.

In some examples, the classification engine accepts one or more input features comprising at least one of presence of a wide jet, severe tricuspid regurgitation, and bileaflet flail prolapse. As an alternative or addition, the classification engine may include one or more weights corresponding to at least one of presence of a wide jet, severe tricuspid regurgitation, and bileaflet flail prolapse. As an alternative or addition, the candidate feature vector may include at least one of presence of a wide jet of the candidate, severe tricuspid regurgitation of the candidate, and bileaflet flail prolapse of the candidate.

In some examples, the classification engine is trained based on input training data comprising patient anatomical features before a mitral TEER procedure for a plurality of patients. As an alternative or addition, the patient anatomical features may include anatomical features determined based on echocardiographic data for the plurality of patients. As an alternative or addition, the patient anatomical features may include a regurgitant volume, an effective regurgitant orifice area, a mitral valve peak velocity, a mean mitral gradient, presence of a wide jet, severe tricuspid regurgitation, and bileaflet flail prolapse for the plurality of patients. As an alternative or addition, the classification engine may be trained based on output training data comprising a determination of mitral TEER success or mitral TEER failure for the plurality of patients. As an alternative or addition, the classification engine may be trained based on output training data generated based on at least one of occurrence of one or more major adverse events after the mitral TEER procedure for the plurality of patients, a mitral gradient after the mitral TEER procedure for the plurality of patients, and a mitral regurgitation grade after the mitral TEER procedure for the plurality of patients.

In some examples, the classification engine is trained based on input training data comprising patient anatomical features after a mitral TEER procedure for a candidate. As an alternative or addition, the patient anatomical features may include anatomical features determined based on echocardiographic data for the candidate taken post-TEER procedure. As an alternative or addition, the patient anatomical features may include a regurgitant volume, an effective regurgitant orifice area, a mitral valve peak velocity, a mean mitral gradient, presence of a wide jet, severe tricuspid regurgitation, and bileaflet flail prolapse for the candidate post-TEER procedure. As an alternative or addition, the classification engine may be trained based on output training data comprising a determination of mitral TEER success or mitral TEER failure for candidate. As an alternative or addition, the classification engine may be trained based on output training data generated based on at least one of occurrence of one or more major adverse events after the mitral TEER procedure for the candidate, a mitral gradient after the mitral TEER procedure for candidate, and a mitral regurgitation grade after the mitral TEER procedure for the candidate.

One aspect of the disclosure is directed to a non-transitory computer-readable medium storing instructions. The instructions, when executed by one or more processors of a network system, cause the network system to receive a set of candidate anatomical features corresponding to a candidate for a mitral TEER procedure; generate a candidate feature vector based on the set of candidate anatomical features; apply a classification engine to the candidate feature vector, wherein the classification engine is trained to evaluate mitral TEER outcome; and provide an outcome metric for the candidate based on applying the classification engine.

In some examples, the classification engine is a binary classification engine comprising a logistic regression model. As an alternative or addition, the outcome metric may correspond to a predicted mitral TEER success or a predicted mitral TEER failure.

In some examples, the classification engine accepts an input feature comprising a first principal component input generated based on a regurgitant volume and an effective regurgitant orifice area. As an alternative or addition, the classification engine may include a first weight corresponding to the first principal component input. As an alternative or addition, the set of candidate anatomical features may include a regurgitant volume of the candidate and an effective regurgitant orifice area of the candidate. As an alternative or addition, the candidate feature vector may include a first principal component input value generated based on the regurgitant volume of the candidate and the effective regurgitant orifice area of the candidate.

In some examples, the classification engine accepts an input feature comprising a second principal component input generated based on a mitral valve peak velocity and a mean mitral gradient. As an alternative or addition, the classification engine may include a second weight corresponding to the second principal component input. As an alternative or addition, the set of candidate anatomical features may include a mitral valve peak velocity of the candidate and a mean mitral gradient of the candidate. As an alternative or addition, the candidate feature vector may include a second principal component input value generated based on the mitral valve peak velocity of the candidate and the mean mitral gradient of the candidate.

In some examples, the classification engine accepts one or more input features comprising at least one of presence of a wide jet, severe tricuspid regurgitation, and bileaflet flail prolapse. As an alternative or addition, the classification engine may include one or more weights corresponding to at least one of presence of a wide jet, severe tricuspid regurgitation, and bileaflet flail prolapse. As an alternative or addition, the candidate feature vector may include at least one of presence of a wide jet of the candidate, severe tricuspid regurgitation of the candidate, and bileaflet flail prolapse of the candidate.

In some examples, the classification engine is trained based on input training data comprising patient anatomical features before a mitral TEER procedure for a plurality of patients. As an alternative or addition, the patient anatomical features may include anatomical features determined based on echocardiographic data for the plurality of patients. As an alternative or addition, the patient anatomical features may include a regurgitant volume, an effective regurgitant orifice area, a mitral valve peak velocity, a mean mitral gradient, presence of a wide jet, severe tricuspid regurgitation, and bileaflet flail prolapse for the plurality of patients. As an alternative or addition, the classification engine may be trained based on output training data comprising a determination of mitral TEER success or mitral TEER failure for the plurality of patients. As an alternative or addition, the classification engine may be trained based on output training data generated based on at least one of occurrence of one or more major adverse events after the mitral TEER procedure for the plurality of patients, a mitral gradient after the mitral TEER procedure for the plurality of patients, and a mitral regurgitation grade after the mitral TEER procedure for the plurality of patients.

In some examples, the classification engine is trained based on input training data comprising patient anatomical features after a mitral TEER procedure for a candidate. As an alternative or addition, the patient anatomical features may include anatomical features determined based on echocardiographic data for the candidate taken post-TEER procedure. As an alternative or addition, the patient anatomical features may include a regurgitant volume, an effective regurgitant orifice area, a mitral valve peak velocity, a mean mitral gradient, presence of a wide jet, severe tricuspid regurgitation, and bileaflet flail prolapse for the candidate post-TEER procedure. As an alternative or addition, the classification engine may be trained based on output training data comprising a determination of mitral TEER success or mitral TEER failure for candidate. As an alternative or addition, the classification engine may be trained based on output training data generated based on at least one of occurrence of one or more major adverse events after the mitral TEER procedure for the candidate, a mitral gradient after the mitral TEER procedure for candidate, and a mitral regurgitation grade after the mitral TEER procedure for the candidate.

In some implementations, the various techniques described herein may achieve one or more of the following advantages: candidate risk assessment and/or screening for a mitral TEER procedure is efficiently performed in a scalable manner; patient outcomes may be improved; a candidate classification model may be iteratively improved based mitral TEER procedure outcomes. Additional features and advantages are apparent from the specification and the drawings.

### System Overview

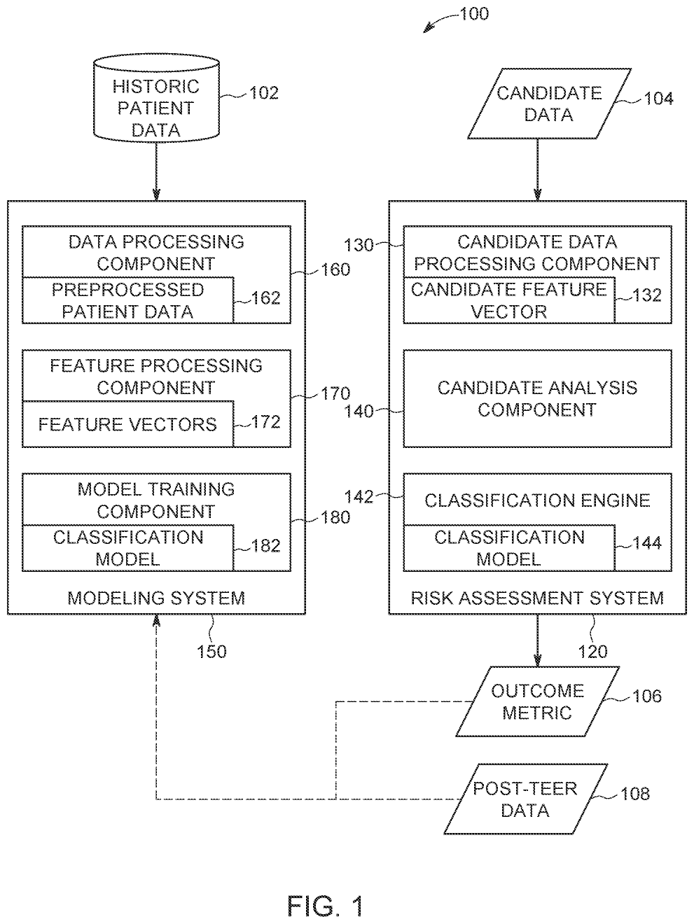

FIG. 1 illustrates a block diagram of an example computer system in accordance with an aspect of the disclosure. The computer system 100 includes a risk assessment system 120 and a modeling system 150. The risk assessment system 120 and/or the modeling system 150 may be communicatively coupled to one or more networks. The network/s may include one or more local area networks (LANs) and/or one or more wide area networks, such as the Internet.

The risk assessment system 120 predicts procedural success for a mitral TEER procedure for one or more candidates for the mitral TEER procedure. The risk assessment system 120 may include a candidate data processing component 130 and a candidate analysis component 140.

The candidate data processing component 130 receives candidate data 104 corresponding to a candidate for a mitral TEER procedure. For example, the candidate may be a patient with primary mitral regurgitation, secondary mitral regurgitation, and/or any other characteristic for which a mitral TEER procedure may be performed. The candidate data 104 may include any data corresponding to the candidate and/or the mitral TEER procedure, such as patient data, physician data, and/or site characteristics. In some examples, the candidate data 104 includes a set of candidate anatomical features. The set of candidate anatomical features may include anatomical features determined based on echocardiographic data collected for the candidate patient. In some examples, the candidate anatomical features may include one or more of a regurgitant volume, an effective regurgitant orifice area, a mitral valve peak velocity, a mean mitral gradient, presence of a wide jet, severe tricuspid regurgitation, and bileaflet flail prolapse for the plurality of patients, and/or other anatomical features of the candidate.

The candidate data processing component 130 processes the candidate data 104 to prepare the candidate data 104 for analysis, such as by generating a candidate feature vector 132 based on the candidate data 104. The candidate feature vector 132 may include any data structure generated based on the candidate data 104 that includes one or more inputs for the classification engine 142. The candidate feature vector 132 includes one or more input features for a classification engine 142. The candidate data processing component 130 may perform consistency checks on the candidate data 104 to ensure that the candidate data 104 includes the necessary data for applying the classification engine 142. As an addition or alternative, the candidate data processing component 130 may format, convert, or otherwise process the candidate data 104 to ensure that the candidate data 104 is in the proper form for applying the classification engine 142.

In some examples, the candidate data processing component 130 applies feature extraction parameters to the candidate data 104 to generate one or more input features, which may be included in the candidate feature vector 132. The feature extraction parameters may be generated by the modeling system 150 that generated a classification model 144, as described in greater detail hereinafter. In some examples, the feature extraction parameters are generated based on principal component analysis. For example, the candidate feature vector 132 may include a first principal component input value generated based on the regurgitant volume of the candidate and the effective regurgitant orifice area of the candidate. As an addition or alternative, the candidate feature vectors 132 may include a second principal component input value generated based on the mitral valve peak velocity of the candidate and the mean mitral gradient of the candidate.

The candidate analysis component 140 applies a classification engine 142 to the candidate feature vector 132. The classification engine 142 is trained to predict a mitral TEER outcome for a candidate based on the candidate feature vector 132 generated based on the candidate data 104. The classification engine 142 may include a classification model 144 corresponding to a classification model 182 generated by the modeling system 150, as described in greater detail hereinafter. In some examples, the classification engine 142 is a binary classification engine that includes a binary classification model, such as but not limited to a logistic regression model. When the classification engine 142 is applied to the candidate feature vector 132, the classification engine 142 generates an output based on the candidate feature vector 132 and the classification model 144.

In some examples, the classification model 144 includes a set of one or more weights corresponding to a set of features included in the candidate feature vector 132. For example, the candidate feature vector 132 may include one or more of a principal component input value generated based on the regurgitant volume of the candidate and the effective regurgitant orifice area of the candidate, a principal component input value generated based on the mitral valve peak velocity of the candidate and a mean mitral gradient of the candidate, an input value corresponding to presence of a wide jet of the candidate, an input value corresponding to severe tricuspid regurgitation of the candidate, and an input value corresponding to bileaflet flail prolapse of the candidate. In some examples, the classification model 144 is a logistic regression model that includes a set of weights corresponding to the set of features included in the candidate feature vector 132.

In some examples, the output of the classification engine 142 is a binary classification. For example, the output of the classification engine 142 may be selected from a predicted mitral TEER success and a predicted mitral TEER failure. As an alternative or addition, the output of the classification engine 142 may be a risk score corresponding to a likelihood of mitral TEER success and/or mitral TEER failure. For example, the output of the classification engine 142 may be a probability, a boolean, an integer, a decimal number, a classification, or any other value that can describe a predicted candidate risk assessment.

After the classification engine 142 is applied, the candidate analysis component 140 provides an outcome metric 106 for the candidate. The outcome metric 106 may be the output of the classification engine 142. For example, when the output of the classification engine 142 is selected from a predicted mitral TEER success and a predicted mitral TEER failure, the output of the classification engine 142 may be used as the outcome metric 106. As an alternative or addition, the outcome metric 106 may be generated based on the output of the classification engine 142. For example, when the output of the classification engine 142 is numeric, the outcome metric 106 may be based on one or more threshold values. The one or more threshold values may include a threshold corresponding to predicted mitral TEER success, a threshold corresponding to predicted mitral TEER failure, a threshold corresponding to a particular confidence level of mitral TEER success, or any other threshold value.

The outcome metric 106 may be provided to a medical specialist treating the candidate to inform treatment decisions for the candidate. For example, if the outcome metric 106 indicates that the candidate is predicted to have a low likelihood of success following a mitral TEER procedure, the medical specialist may use the outcome metric 106 in determining whether to pursue alternative treatment pathways for the candidate. As another example, if the outcome metric 106 indicates that the candidate is predicted to have a high likelihood of success following a mitral TEER procedure, the medical specialist may use the outcome metric 106 in determining whether to recommend the mitral TEER procedure for the candidate.

The risk assessment system 120 and/or its components (e.g. candidate data processing component 130, candidate analysis component 140, and/or classification engine 142) as described herein are presented as individual components for case of explanation. Any action performed by or to one or more components of the risk assessment system 120 may be considered performed by or to the risk assessment system 120. The risk assessment system 120 and/or its components may be implemented as one or more dependent or independent processes, and may be implemented on one or multiple computers. For example, a component may be implemented as a distributed system. As an alternative or addition, multiple instances of one or more components may be implemented. In some examples, one or more components of the risk assessment system 120 may be implemented as a cloud service and/or using one or more cloud service providers. Furthermore, a component may be implemented fully and/or partially in one or multiple programs and/or processes, and two or more components shown may be implemented fully and/or partially in the same program and/or process.

The modeling system 150 generates a classification model 182 that predicts a candidate's outcome for a mitral TEER procedure. The modeling system 150 may include a data processing component 160, a feature processing component 170, and/or a model training component 180.

The data processing component 160 processes historic patient data 102 for a plurality of patients that have undergone a mitral TEER procedure to generate preprocessed patient data 162. The data processing component 160 may detect missing data, verify data, categorize data, normalize data, or otherwise preprocess the historic patient data 102 to generate the preprocessed patient data 162. The preprocessed patient data 162 includes a plurality of features for each patient of the plurality of patients. A feature refers to an individual value corresponding to a property or characteristic.

The historic patient data 102 may include patient data associated with individual patients of the plurality of patients, physician data associated with a mitral TEER procedure, and/or site characteristics associated with a mitral TEER procedure. In some examples, the plurality of patients are participants in a patient registry associated with mitral regurgitation and/or mitral TEER. The patient data may include any patient data, such as but not limited to demographic data, medical measurements, laboratory data, or any other medical data. The historic patient data 102 may be collected from one or more data sources, such as but not limited to medical records, health records, medical billing data, registry data, or other data sources.

In some examples, the historic patient data 102 includes anatomical features determined before a mitral TEER procedure for a plurality of patients, such as but not limited to anatomical features determined based on echocardiographic data and/or other imaging data corresponding to the individual patients of the plurality of patients. In some examples, the historic patient data 102 includes anatomical features determined based on echocardiographic data collected before a mitral TEER procedure, upon discharge after a mitral TEER procedure, 30-days after a mitral TEER procedure, and 1 year after a mitral TEER procedure. The anatomical features collected via echocardiogram may include one or more of mitral regurgitation severity grade, effective regurgitant orifice area (EROA), coaptation length and depth, flail measures (gap/width), grasping area anatomy, regurgitant jet(s) position and quantity, and tricuspid regurgitation severity amongst others.

The feature processing component 170 processes the preprocessed patient data 162 to identify features for training the classification model 182. In some examples, the identification of features for training the classification model 182 may involve training and/or machine learning techniques, which may be iteratively performed.

In some examples, the feature processing component 170 performs feature selection based on the preprocessed patient data 162. The preprocessed patient data 162 includes a plurality of features for each patient of the plurality of patients. Feature selection identifies a set of features to be used to train the classification model 182. For example, the initial set of features contained in the preprocessed patient data 162 may be redundant and/or too numerous.

In some examples, the feature processing component 170 performs feature extraction based on the preprocessed patient data 162. Feature extraction generates one or more derived features from the initial set of features. Feature extraction techniques may be used to reduce a set of features to be used as training data, such as when two or more features are highly correlated. In some examples, the feature processing component 170 uses principal component analysis (PCA) techniques to generate one or more derived features. For example, the feature processing component 170 may generate a derived feature based on a regurgitant volume feature and an effective regurgitant orifice area feature. As another example, the feature processing component 170 may generate a derived feature based on a mitral valve peak velocity feature and a mean mitral gradient feature. The feature processing component 170 may generate feature extraction parameters that indicate how to generate feature values for one or more derived features based on feature values for the underlying features. For example, the feature extraction parameters corresponding to one or more derived features may be used by the candidate data processing component 130 to generate a candidate feature vector 132 from candidate data 104.

The feature processing component 170 may generate a plurality of feature vectors 172 for use by the model training component 180. Each feature vector of the plurality of feature vectors 172 includes selected features and/or derived features for a patient of the plurality of patients corresponding to features of the patient before the mitral TEER procedure was performed.

In some examples, the feature processing component 170 tags the plurality of feature vectors 172 with training output data. For example, a particular feature corresponding to a patient that has undergone a mitral TEER procedure may be tagged with an output value, such as a mitral TEER success and/or a mitral TEER failure. The historic patient data 102 may include anatomical features determined based on echocardiographic data collected after the mitral TEER procedure, which may be used in tagging the plurality of feature vectors with an outcome (e.g., mitral TEER success, mitral TEER failure). For example, a determination of mitral TEER success and/or mitral TEER failure may be based on the occurrence of at least one major adverse event after the mitral TEER procedure, a mitral gradient <8 mmHg after the mitral TEER procedure, and a mitral regurgitation grade ≤1 after the mitral TEER procedure.

The model training component 180 performs one or more machine learning techniques to generate and/or adjust a classification model 182. In some examples, the model training component 180 iteratively trains and/or tests the classification model 182 using supervised learning techniques. For example, iteratively training the classification model 182 may include adjusting one or more weights of the classification model 182. In some examples, the classification model 182 is a logistic regression model that includes a weight for each input feature of the feature vectors 172 for the plurality of patients. The model training component 180 may continue to train the classification model 182 until a level of performance is optimized. As an alternative and/or addition, the model training component 180 may test and/or validate the classification model using test training data and/or validation training data set aside for the testing and/or validation.

The modeling system 150 and/or its components (e.g., data processing component 160, feature processing component 170, and/or model training component 180) as described herein are presented as individual components for case of explanation. Any action performed by or to one or more components of the modeling system 150 may be considered performed by or to the modeling system 150. The modeling system 150 and/or its components may be implemented as one or more dependent or independent processes, and may be implemented on one or multiple computers. For example, a component may be implemented as a distributed system. As an alternative or addition, multiple instances of one or more components may be implemented. In some examples, one or more components of the modeling system 150 may be implemented as a cloud service and/or using one or more cloud service providers. Furthermore, a component may be implemented fully and/or partially in one or multiple programs and/or processes, and two or more components shown may be implemented fully and/or partially in the same program and/or process.

The risk assessment system 120 and the modeling system 150 are illustrated as distinct computer systems. In some examples, the risk assessment system 120 and the modeling system 150 may be fully distinct, partially overlapping, or the same computer system.

In some examples, the model training component 180 updates the classification model 182 based on the outcome of one or more candidates processed by the risk assessment system 120. For example, the outcome metric 106 and post-TEER data 108 corresponding to one or more candidates that have undergone a mitral TEER procedure may be used to adjust the classification model 182.

### Example Logistic Regression Model

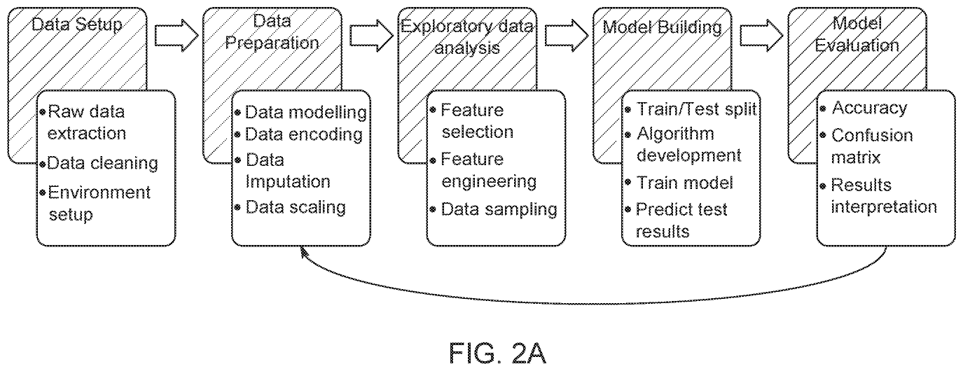

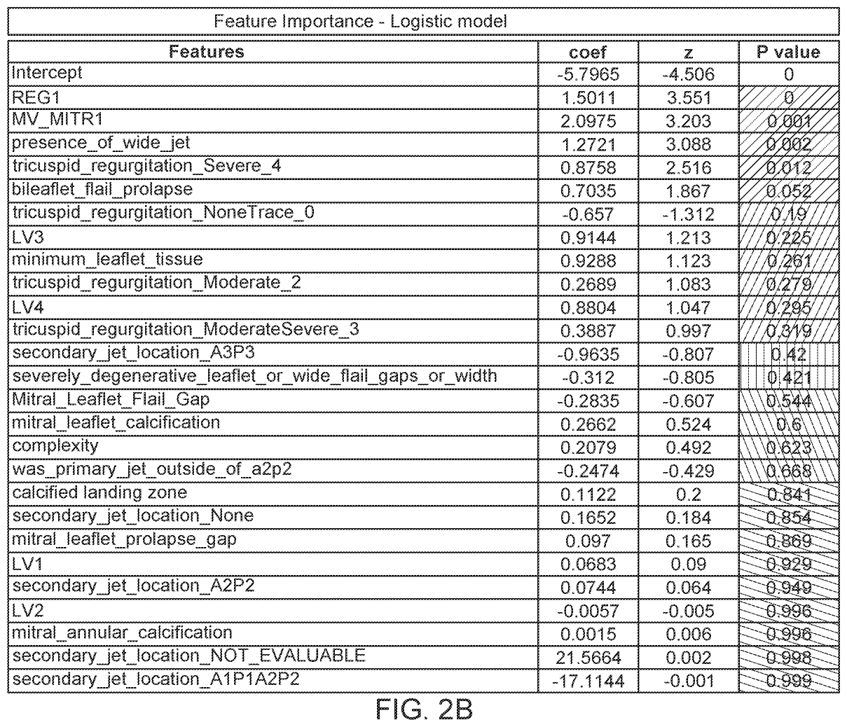

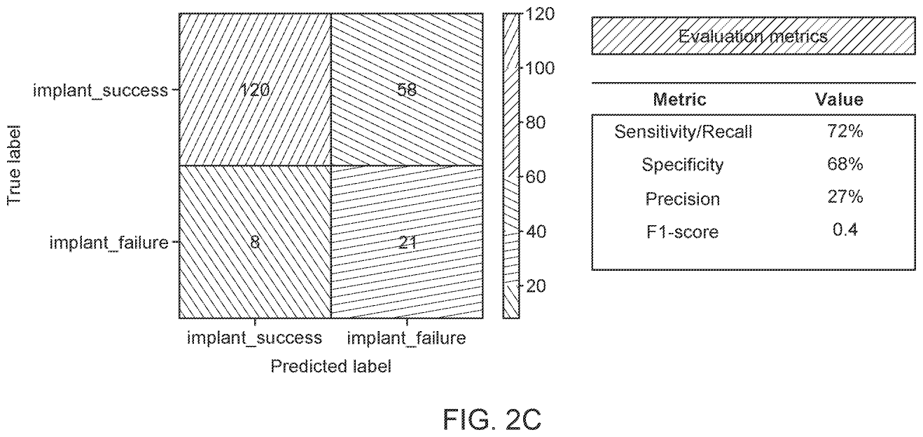

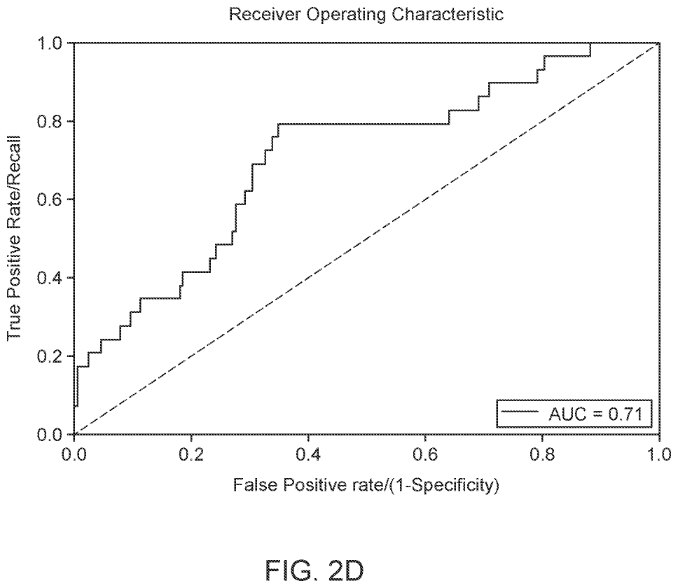

An example risk assessment system performs candidate risk assessment for a mitral TEER procedure. FIG. 2A illustrates a block diagram of an example process for building, training, and evaluating a classification model. FIG. 2B illustrates example feature selection data for a logistic regression model including principal component features. FIG. 2C illustrates example evaluation data for a logistic regression model. FIG. 2D illustrates example evaluation data for a logistic regression model.

A logistic regression model was built using anatomical data for patients with primary MR in Abbott's EXPAND and EXPAND G4 registries. Data was obtained from the Abbott EXPAND and EXPAND G4 registries, which are Abbott-sponsored prospective, multi-center, single-arm observational studies. The objective of these registries is to assess the safety and performance of the Abbott mitral TEER system (MitraClip™) in a post-market real-world setting.

The model required advanced analytic methods including data imputation by clinical and statistical techniques, principal component analysis, up-sampling, and regularization. Alternative models such as support vector machine (SVM), KNN, Random Forest, and XG Boost Dynamic Selection were also evaluated. The final model achieved a sensitivity of 72%, specificity of 68%, precision at 27% and F1-score of 0.4 in predicting procedural success of mitral TEER.

Echocardiographic data is collected from enrolled patients at baseline, discharge, 30-days, and 1 year post-procedure. Patient anatomical features collected via echocardiogram include MR severity grade, effective regurgitant orifice area (EROA), coaptation length and depth, flail measures (gap/width), grasping area anatomy, regurgitant jet(s) position and quantity, and tricuspid regurgitation severity amongst others. Major adverse events (MAE) are collected throughout study follow-up and include all-cause mortality, myocardial infarction, stroke, or mitral valve reintervention. Additional information on the registries is included in the table below. Patient data from EXPAND and EXPAND G4 was combined and used to build the case classification prediction model.

To meet the definition of procedural success, all three of the following conditions were required to be met at 30 days post mitral TEER: no MAE, mitral gradient <8 mmHg, and MR grade ≤1. If any one of the following conditions were met at 30 days, the procedure was defined as not successful: ≥1 MAE, mitral gradient ≥8 mmHg, or MR>1+. Patients with missing data for any of these criteria were excluded from the analysis if the other criteria for success were not met.

Building the model to predict procedural success required use of advanced data science techniques. Implementing these data science techniques was uniquely challenging due to several key limitations of the available data in the EXPAND and EXPAND G4 registries.

First, several of the input parameters had significant amounts of missing data that needed to be imputed to build a workable model. Twenty-seven anatomical parameters were collected from registry data and recommended by clinicians for inclusion as inputs to the model. 14 of these variables had significant amounts of missing data. Missing data were imputed using various clinical and statistical techniques depending on the variable. Clinical techniques include collecting site-reported echocardiography values and statistical techniques include K Nearest Neighbors (KNN), conditional KNN and mean values.

Another challenge with the input parameters was multicollinearity among several of these variables. To address this issue, feature engineering, specifically Principal Component Analysis (PCA) was used to group correlated variables. Groupings were confirmed based on clinical judgment.

The third key challenge with available data was the small sample size (826 patients), which makes modeling more difficult, particularly when building a model using several input parameters (dimensions). Further complicating this issue was the lack of balanced buckets among the sample: very few patients in the registry data had a procedural failure, meaning the success bucket was a much larger percentage of the sample than the failure bucket. This issue was mitigated in two ways. First, the target variables were combined (as described above, success was defined as meeting all three of the following conditions: no MAE, mitral gradient <8 mmHg, and MR grade ≤1) because together, the variables had slightly more balanced buckets than each target variable on its own. Second, in the training dataset, the procedural failure bucket was randomly up-sampled to increase sample size, thereby lessening the imbalance.

The fourth challenge was how to identify the best model to predict procedural failure. Multiple modeling techniques were employed, including logistic regression, support vector machine (SVM), KNN, Random Forest, and XG Boost Dynamic Selection. Logistic regression achieved the highest accuracy among these techniques.

The fifth and final challenge encountered in the modeling is what is referred to as the curse of dimensionality. As noted above, the sample size was small (n=826) and the number of input parameters, i.e., dimensions, relatively large (27). This led to issues with overfitting: the model was overfit to the training data. Various techniques were used to confirm that overfitting was occurring and that the most predictive variables were selected (e.g., forward stepwise regression with backward elimination, comparing the base model to a regularized model).

The final model consisted of a logistic regression with the target outcome defined as the probability of procedural failure and five input variables with p-values <0.05: (i) PCA component 1, which included regurgitant volume and effective regurgitant orifice area; (ii) PCA component 2, which included mitral valve peak velocity and mean mitral gradient; (iii) presence of a wide jet; (iv) severe tricuspid regurgitation; and (v) bileaflet flail prolapse. This model achieved a sensitivity of 72%, specificity of 68%, precision at 27% and F1-score of 0.4.

### Example Processes

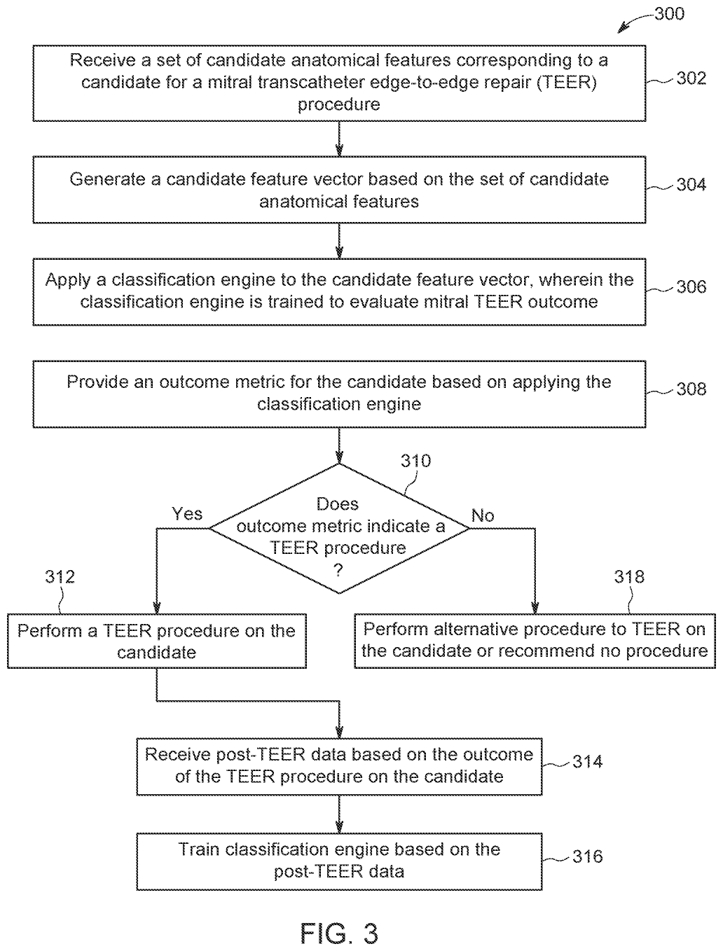

FIG. 3 illustrates a flow diagram of an example process for candidate risk assessment for a mitral TEER procedure. Process 300 may be performed by one or more computing devices and/or processes thereof. For example, one or more blocks of process 300 may be performed by a computer system, such as but not limited to computer system 400. Process 300 will be described with respect to the risk assessment system 120 of FIG. 1, but is not limited to performance by such.

At block 302, the risk assessment system 120 receives a set of candidate anatomical features corresponding to a candidate for a mitral TEER procedure. In some examples, the set of candidate anatomical features includes anatomical features determined based on echocardiographic data collected for the candidate. As an addition, the candidate anatomical features may include one or more of a regurgitant volume, an effective regurgitant orifice area, a mitral valve peak velocity, a mean mitral gradient, presence of a wide jet, severe tricuspid regurgitation, and bileaflet flail prolapse for the plurality of patients, and/or other anatomical features of the candidate.

At block 304, the risk assessment system 120 generates a candidate feature vector based on the set of candidate anatomical features. The candidate feature vector may include one or more input values, such as but not limited to a first principal component input value generated based on the regurgitant volume of the candidate and the effective regurgitant orifice area of the candidate, a second principal component input value generated based on the mitral valve peak velocity of the candidate and the a mean mitral gradient of the candidate, presence of a wide jet of the candidate, severe tricuspid regurgitation of the candidate, and/or bileaflet flail prolapse of the candidate.

At block 306, the risk assessment system 120 applies a classification engine to the candidate feature vector, wherein the classification engine is trained to evaluate mitral TEER outcome. In some examples, the classification engine is a binary classification engine comprising a logistic regression model. In some examples, the classification engine is trained based on input training data comprising patient anatomical features before a mitral TEER procedure for a plurality of patients. The patient anatomical features may include anatomical features determined based on echocardiographic data for the plurality of patients. As an alternative or addition, the patient anatomical features may include a regurgitant volume, an effective regurgitant orifice area, a mitral valve peak velocity, a mean mitral gradient, presence of a wide jet, severe tricuspid regurgitation, and bileaflet flail prolapse, and/or other anatomical features of the set of patients. As an alternative or addition, the classification engine may be trained based on output training data comprising a determination of mitral TEER success or mitral TEER failure for the plurality of patients. As an alternative or addition, the classification engine may be trained based on output training data generated based on at least one of occurrence of one or more major adverse events after the mitral TEER procedure for the plurality of patients, a mitral gradient after the mitral TEER procedure for the plurality of patients, and a mitral regurgitation grade after the mitral TEER procedure for the plurality of patients. In some examples, the classification engine accepts an input feature comprising a first principal component input generated based on a regurgitant volume and an effective regurgitant orifice area. The classification engine may include a first weight corresponding to the first principal component input. As an alternative or addition, the classification engine may accept an input feature comprising a second principal component input generated based on a mitral valve peak velocity and a mean mitral gradient. The classification engine may include a second weight corresponding to the second principal component input. As an alternative or addition, the classification engine may accept one or more input features comprising at least one of presence of a wide jet, severe tricuspid regurgitation, and bileaflet flail prolapse. The classification engine may include one or more weights corresponding to at least one of presence of a wide jet, severe tricuspid regurgitation, and bileaflet flail prolapse.

At block 308, the risk assessment system 120 provides an outcome metric for the candidate based on applying the classification engine. In some examples, the outcome metric corresponds to a predicted mitral TEER success or a predicted mitral TEER failure.

At block 310, where the outcome metric 106 meets a threshold level to qualify as a predicted TEER success, the outcome metric may indicate a TEER procedure. Alternatively, where the outcome metric 106 may instead predict mitral TEER failure, then a TEER procedure may be contraindicated.

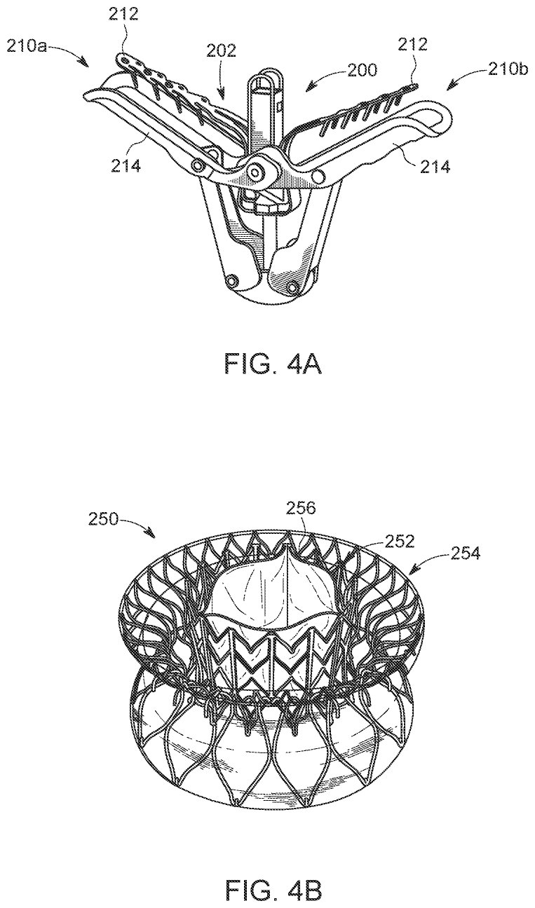

At block 312, where a TEER procedure may be indicated based on the outcome metric 106, a TEER procedure may then be performed with an implantable fixation device, for example. An exemplary implantable fixation device 210 is depicted in FIG. 4A and may generally include a central assembly 202 and a pair of clamps 210a, 210b coupled to the central assembly 202. The pair of clamps 210a, 210b may each be comprised of a proximal element 212 and a distal element 214. The proximal and distal elements 212, 214 of each clamp 210a. 210b may be moveable relative to each other to grasp and secure valve leaflet tissue therebetween. In this regard, a TEER procedure may include delivering the implantable fixation device 200 (e.g., percutaneously) to a mitral valve and deploying the implantable fixation device 200 such that the first clamp 210a grasps a first leaflet, and the second clamp 210b grasps a second valve leaflet to improve coaptation, such as though the formation of a double orifice, for example.

At block 314, post-TEER data 108 based on the outcome of the TEER procedure on the candidate patient may be received by the modeling system 150, as illustrated in FIG. 1. Such post-TEER data 108 may include the binary assessment of mitral TEER success or mitral TEER failure for the candidate. Other post-TEER data 108 may be indicative of the success of the TEER procedure and may therefore confirm the prediction of the outcome metric 106 as being correct or incorrect. For example, post-TEER data 108 can also include the occurrence(s) of one or more adverse events, mitral gradient after the mitral TEER procedure for the candidate, and mitral regurgitation grade after the mitral TEER procedure for the candidate. Other post-TEER data 108 can include a regurgitant volume, an effective regurgitant orifice area, a mitral valve peak velocity, a mean mitral gradient, presence of a wide jet post-TEER, severe tricuspid regurgitation, and bileaflet flail prolapse each of which may be utilized by the modeling system 150 in its training data and as an apples-to-apples comparison with the same measurements obtained prior to the TEER procedure. Some or all of the post-TEER data may be obtained through echocardiographic evaluation or through other diagnostic tests, for example.

At block 316, the post-TEER data 108 may be used to train the classification model 182. As such, the classification model 182 may be a supervised machine learning model. Training of the classification model 182 with the post-TEER data may include adjusting one or more weights of the classification model 182. Also, as shown in FIG. 1, the outcome metric 106 may also be received within modeling system 150 and may provide further training data for classification model modification.

At block 318, in the event a TEER procedure is contraindicated based on the outcome metric predicting TEER failure, an alternative procedure to mitral TEER may be performed or it may be recommended that no procedure be immediately performed. An alternative procedure may include mitral valve replacement with a mitral valve prosthesis, for example. An exemplary mitral valve prosthesis 250 is depicted in FIG. 4B and may generally include a strut frame 252, an anchor assembly 256, and a plurality of replacement valve leaflets 256. In the example depicted, anchor assembly 254 and replacement leaflets 256 may be coupled to the strut frame 252 such that mitral valve prosthesis 250 is collapsible and expandable. Mitral valve prosthesis 250 may be guided to the mitral valve (e.g., percutaneously) and deployed therein such that the anchor assembly 254 may be anchored to the left atrium and the left ventricle of the heart. Other alternative procedures, such as an annuloplasty, for example, may be performed in lieu of a mitral TEER procedure where a TEER procedure is contraindicated. The outcome metric 106 provided by the risk assessment system 120 may help determine which alternative procedure to perform and/or the risk assessment system 120 may provide a recommendation based on the outcome metric 106.

### Implementation Mechanisms—Hardware Overview

The techniques described herein may be implemented by one or more special-purpose computing devices. The special-purpose computing devices may be hard-wired to perform one or more techniques described herein, including combinations thereof. Alternatively and/or in addition, the one or more special-purpose computing devices may include digital electronic devices such as one or more application-specific integrated circuits (ASICs) or field-programmable gate arrays (FPGAs) that are persistently programmed to perform the techniques. Alternatively and/or in addition, the one or more special-purpose computing devices may include one or more general-purpose hardware processors programmed to perform the techniques described herein pursuant to program instructions in firmware, memory, other storage, or a combination. Such special-purpose computing devices may also combine custom hard-wired logic, ASICs, or FPGAs with custom programming to accomplish the techniques. The special-purpose computing devices may be desktop computer systems, portable computer systems, handheld devices, networking devices, and/or any other device that incorporates hard-wired or program logic to implement the techniques.

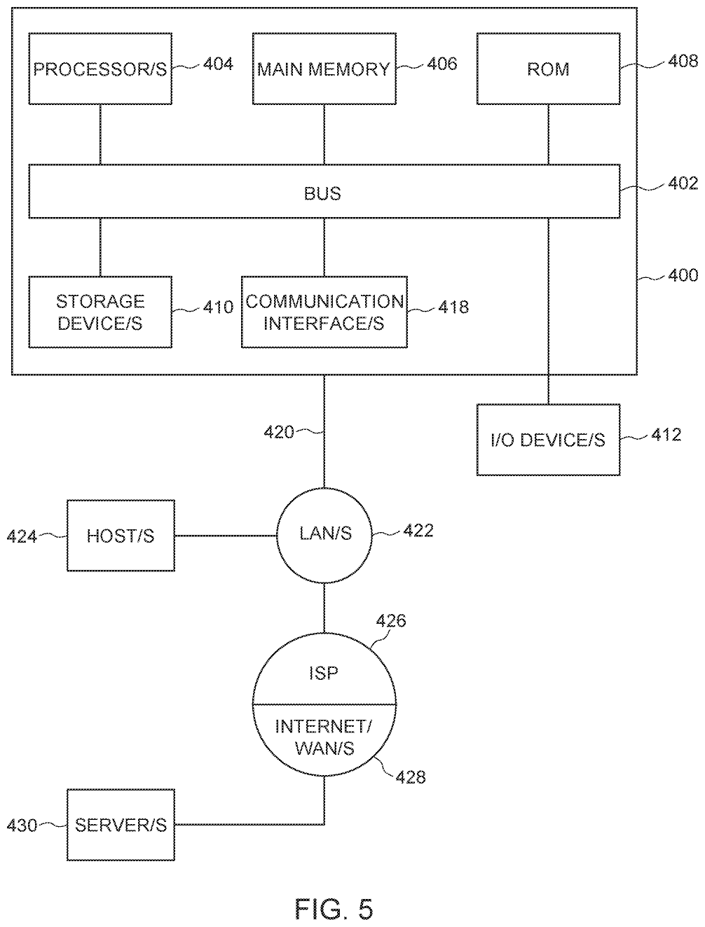

FIG. 5 is a block diagram that illustrates a computer system upon which an example may be implemented. The computer system 400 includes a bus 402 or other communication mechanism for communicating information, and one or more hardware processors 404 coupled with bus 402 for processing information, such as computer instructions and data. The processor/s 404 may include one or more general-purpose microprocessors, graphical processing units (GPUs), coprocessors, central processing units (CPUs), and/or other hardware processing units.

The computer system 400 also includes one or more units of main memory 406 coupled to the bus 402, such as random-access memory (RAM) or other dynamic storage, for storing information and instructions to be executed by the processor/s 404. Main memory 406 may also be used for storing temporary variables or other intermediate information during execution of instructions to be executed by the processor/s 404. Such instructions, when stored in non-transitory storage media accessible to the processor/s 404, turn the computer system 400 into a special-purpose machine that is customized to perform the operations specified in the instructions. In some embodiments, main memory 406 may include dynamic random-access memory (DRAM) (including but not limited to double data rate synchronous dynamic random-access memory (DDR SDRAM), thyristor random-access memory (T-RAM), zero-capacitor (Z-RAM™)) and/or non-volatile random-access memory (NVRAM).

The computer system 400 may further include one or more units of read-only memory (ROM) 408 or other static storage coupled to the bus 402 for storing information and instructions for the processor/s 404 that are either always static or static in normal operation but reprogrammable. For example, the ROM 408 may store firmware for the computer system 400. The ROM 408 may include mask ROM (MROM) or other hard-wired ROM storing purely static information, programmable read-only memory (PROM), erasable programmable read-only memory (EPROM), electrically-erasable programmable read-only memory (EEPROM), another hardware memory chip or cartridge, or any other read-only memory unit.

One or more storage devices 410, such as a magnetic disk or optical disk, is provided and coupled to the bus 402 for storing information and/or instructions. The storage device/s 410 may include non-volatile storage media such as, for example, read-only memory, optical disks (such as but not limited to compact discs (CDs), digital video discs (DVDs), Blu-ray discs (BDs)), magnetic disks, other magnetic media such as floppy disks and magnetic tape, solid-state drives, flash memory, optical disks, one or more forms of non-volatile random-access memory (NVRAM), and/or other non-volatile storage media.

The computer system 400 may be coupled via the bus 402 to one or more input/output (I/O) devices 412. For example, the I/O device/s 412 may include one or more displays for displaying information to a computer user, such as a cathode ray tube (CRT) display, a Liquid Crystal Display (LCD) display, a Light-Emitting Diode (LED) display, a projector, and/or any other type of display.

The I/O device/s 412 may also include one or more input devices, such as an alphanumeric keyboard and/or any other keypad device. The one or more input devices may also include one or more cursor control devices, such as a mouse, a trackball, a touch input device, or cursor direction keys for communicating direction information and command selections to the processor 404 and for controlling cursor movement on another I/O device (e.g. a display). A cursor control device typically has degrees of freedom in two or more axes, (e.g. a first axis x, a second axis y, and optionally one or more additional axes z), that allows the device to specify positions in a plane. In some embodiments, the one or more I/O device/s 412 may include a device with combined I/O functionality, such as a touch-enabled display.

Other I/O device/s 412 may include a fingerprint reader, a scanner, an infrared (IR) device, an imaging device such as a camera or video recording device, a microphone, a speaker, an ambient light sensor, a pressure sensor, an accelerometer, a gyroscope, a magnetometer, another motion sensor, or any other device that can communicate signals, commands, and/or other information with the processor/s 404 over the bus 402.

The computer system 400 may implement the techniques described herein using customized hard-wired logic, one or more ASICs or FPGAs, firmware, and/or program logic which, in combination with the computer system causes or programs, causes computer system 400 to be a special-purpose machine. In some examples, the techniques herein are performed by the computer system 400 in response to the processor/s 404 executing one or more sequences of one or more instructions contained in main memory 406. Such instructions may be read into main memory 406 from another storage medium, such as the one or more storage device/s 410. Execution of the sequences of instructions contained in main memory 406 causes the processor/s 404 to perform the process steps described herein. In alternative embodiments, hard-wired circuitry may be used in place of or in combination with software instructions.

The computer system 400 also includes one or more communication interfaces 418 coupled to the bus 402. The communication interface/s 418 provide two-way data communication over one or more physical or wireless network links 420 that are connected to a local network 422 and/or a wide area network (WAN), such as the Internet. For example, the communication interface/s 418 may include an integrated services digital network (ISDN) card, cable modem, satellite modem, or a modem to provide a data communication connection to a corresponding type of telephone line. Alternatively and/or in addition, the communication interface/s 418 may include one or more of: a local area network (LAN) device that provides a data communication connection to a compatible local network 422; a wireless local area network (WLAN) device that sends and receives wireless signals (such as electrical signals, electromagnetic signals, optical signals or other wireless signals representing various types of information) to a compatible LAN; a wireless wide area network (WWAN) device that sends and receives such signals over a cellular network; and other networking devices that establish a communication channel between the computer system 400 and one or more LANs 422 and/or WANs.

The network link/s 420 typically provides data communication through one or more networks to other data devices. For example, the network link/s 420 may provide a connection through one or more local area networks 422 (LANs) to one or more host computers 424 or to data equipment operated by an Internet Service Provider (ISP) 426. The ISP 426 provides connectivity to one or more wide area networks 428, such as the Internet. The LAN/s 422 and WAN/s 428 use electrical, electromagnetic, or optical signals that carry digital data streams. The signals through the various networks and the signals on the network link/s 420 and through the communication interface/s 418 are example forms of transmission media, or transitory media.

The term “storage media” as used herein refers to any non-transitory media that stores data and/or instructions that cause a machine to operate in a specific fashion. Such storage media may include volatile and/or non-volatile media. Storage media is distinct from but may be used in conjunction with transmission media. Transmission media participates in transferring information between storage media. For example, transmission media includes coaxial cables, copper wire and fiber optics, including traces and/or other physical electrically conductive components that comprise the bus 402. Transmission media can also take the form of acoustic or light waves, such as those generated during radio-wave and infra-red data communications.

Various forms of media may be involved in carrying one or more sequences of one or more instructions to the processor 404 for execution. For example, the instructions may initially be carried on a magnetic disk or solid-state drive of a remote computer. The remote computer can load the instructions into its main memory 406 and send the instructions over a telecommunications line using a modem. A modem local to the computer system 400 can receive the data on the telephone line and use an infra-red transmitter to convert the data to an infra-red signal. An infra-red detector can receive the data carried in the infra-red signal and appropriate circuitry can place the data on the bus 402. The bus 402 carries the data to main memory 406, from which the processor 404 retrieves and executes the instructions. The instructions received by main memory 406 may optionally be stored on the storage device 410 either before or after execution by the processor 404.

The computer system 400 can send messages and receive data, including program code, through the network(s), the network link 420, and the communication interface/s 418. In the Internet example, one or more servers 430 may transmit signals corresponding to data or instructions requested for an application program executed by the computer system 400 through the Internet 428, ISP 426, local network 422 and a communication interface 418. The received signals may include instructions and/or information for execution and/or processing by the processor/s 404. The processor/s 404 may execute and/or process the instructions and/or information upon receiving the signals by accessing main memory 406, or at a later time by storing them and then accessing them from the storage device/s 410.

### OTHER ASPECTS OF DISCLOSURE

Although the concepts herein have been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

1. A computer-implemented method comprising:
receiving a set of candidate anatomical features corresponding to a candidate for a mitral transcatheter edge-to-edge repair (TEER) procedure;
generating a candidate feature vector based on the set of candidate anatomical features;
applying a classification engine to the candidate feature vector, wherein the classification engine is trained to evaluate mitral TEER outcome; and
providing an outcome metric corresponding to a predicted mitral TEER success or a predicted mitral TEER failure for the candidate based on applying the classification engine;
wherein the method is performed by one or more processors.

2. The computer-implemented method of claim 1, wherein the classification engine is a binary classification engine comprising a logistic regression model

3. The computer-implemented method of claim 1:
wherein the classification engine accepts an input feature comprising a first principal component input generated based on a regurgitant volume and an effective regurgitant orifice area;
wherein the classification engine includes a first weight corresponding to the first principal component input;
wherein the set of candidate anatomical features includes a regurgitant volume of the candidate and an effective regurgitant orifice area of the candidate; and
wherein the candidate feature vector includes a first principal component input value generated based on the regurgitant volume of the candidate and the effective regurgitant orifice area of the candidate.

4. The computer-implemented method of claim 1:
wherein the classification engine accepts an input feature comprising a second principal component input generated based on a mitral valve peak velocity and a mean mitral gradient;
wherein the classification engine includes a second weight corresponding to the second principal component input;
wherein the set of candidate anatomical features includes a mitral valve peak velocity of the candidate and a mean mitral gradient of the candidate; and
wherein the candidate feature vector includes a second principal component input value generated based on the mitral valve peak velocity of the candidate and the mean mitral gradient of the candidate.

5. The computer-implemented method of claim 1:
wherein the classification engine accepts one or more input features comprising at least one of presence of a wide jet, severe tricuspid regurgitation, and bileaflet flail prolapse;
wherein the classification engine includes one or more weights corresponding to at least one of presence of a wide jet, severe tricuspid regurgitation, and bileaflet flail prolapse; and
wherein the candidate feature vector includes at least one of presence of a wide jet of the candidate, severe tricuspid regurgitation of the candidate, and bileaflet flail prolapse of the candidate.

6. The computer-implemented method of claim 1, wherein the classification engine is trained based on input training data comprising patient anatomical features before a mitral TEER procedure for a plurality of patients.

7. The computer-implemented method of claim 6, wherein the patient anatomical features include anatomical features determined based on echocardiographic data for the plurality of patients.

8. The computer-implemented method of claim 6, wherein the patient anatomical features include a regurgitant volume, an effective regurgitant orifice area, a mitral valve peak velocity, a mean mitral gradient, presence of a wide jet, severe tricuspid regurgitation, and bileaflet flail prolapse for the plurality of patients.

9. The computer-implemented method of claim 6, wherein the classification engine is trained based on output training data comprising a determination of mitral TEER success or mitral TEER failure for the plurality of patients.

10. The computer-implemented method of claim 6, wherein the classification engine is trained based on output training data generated based on at least one of occurrence of one or more major adverse events after the mitral TEER procedure for the plurality of patients, a mitral gradient after the mitral TEER procedure for the plurality of patients, and a mitral regurgitation grade after the mitral TEER procedure for the plurality of patients.

11. A method for treating mitral valve regurgitation, comprising:
performing the computer implemented method according to claim 1;
if the outcome metric predicts mitral TEER success, performing a TEER procedure on the candidate, the TEER procedure comprising:
delivering an implantable fixation device to the mitral valve; and
grasping first and second leaflets of the mitral valve with respective first and second clamps of the implantable fixation device.

12. The method of treatment of claim 11, wherein
if the outcome metric predicts mitral TEER failure, performing an alternative procedure on the candidate, the alternative procedure comprising:
securing a mitral valve prosthesis at least within an annulus of the mitral valve.

13. The method of treatment of claim 12, wherein the securing step includes:
positioning the mitral valve prosthesis having a plurality of replacement valve leaflets and a strut frame within at least the mitral valve annulus, and
deploying the mitral valve prosthesis by expanding the strut frame and the plurality of replacement valve leaflets coupled thereto.

14. The method of treatment of claim 11, wherein the step of performing the TEER procedure further includes:
receiving, at the processor, post-TEER data based on an outcome of the TEER procedure, and
updating, via the processor, the classification engine based on the post-TEER data.

15. The method of treatment of claim 14, wherein the post-TEER data of the candidate includes at least one of the occurrence of one or more major adverse events, a mitral valve gradient, a mitral regurgitation grade, and mitral TEER success or failure.

16. A method for treating mitral valve regurgitation, comprising:
performing the computer implemented method according to claim 1;
if the outcome metric predicts mitral TEER failure, performing an alternative procedure on the candidate, the alternative procedure comprising:
securing a mitral valve prosthesis at least within an annulus of the mitral valve.

17. The method of treatment of claim 16, wherein performing the alternative procedure includes:
positioning the mitral valve prosthesis having a plurality of replacement valve leaflets and a strut frame within at least the mitral valve annulus, and
deploying the mitral valve prosthesis by expanding the strut frame and the plurality of replacement valve leaflets coupled thereto

18. The method of treatment of claim 16, wherein
if the outcome metric predicts mitral TEER success, performing a TEER procedure on the candidate, the TEER procedure comprising:
delivering an implantable fixation device to the mitral valve; and
grasping first and second leaflets of the mitral valve with respective first and second clamps of the implantable fixation device.

19. The method of treatment of claim 18, wherein the step of performing the TEER procedure further includes:
receiving, at the processor, post-TEER data based on an outcome of the TEER procedure, and
updating, via the processor, the classification engine based on the post-TEER data.

20. The method treatment of claim 18, wherein the post-TEER data of the candidate includes at least one of the occurrence of one or more major adverse events, a mitral valve gradient, a mitral regurgitation grade, and mitral TEER success or failure
